(19) United States
(12) Patent Application Publication
(10) Pub. No.: US 2026/0272304 A1
(43) Pub. Date: Sep. 17, 2026

(54) Device and system for therapeutic or diagnostic illumination of intracorporeal parts of a body

(71) Applicant: József KORCSIK, Budapest (HU)

(72) Inventor: József KORCSIK, Budapest (HU)

(21) Appl. No.: 19/427,909

(22) Filed: Dec. 19, 2025

Related U.S. Application Data
(60) Provisional application No. 63/771,809, filed on Mar. 14, 2025.

(51) Int. Cl. A61B 5/00; A61N 5/06

(52) U.S. Cl. CPC A61B 5/0059; A61B 5/0022; A61B 5/4836; A61N 5/0621; A61B 2503/045; A61B 2560/0214; A61B 2560/0276; A61N 2005/0627

(57) ABSTRACT

Provided herein are embodiments relating to devices for clinical treatment and phototherapy of intracorporeal organs or tissues, systems for therapeutic and diagnostic illumination of intracorporeal parts of a body and methods of using such devices.

This application claims the benefit of US Provisional Patent Application No. 63/771,809 filed Mar. 14, 2025, the contents of which are incorporated herein by reference in their entirety.

### FIELD OF THE INVENTION

The present invention relates to devices, systems, and methods for therapeutic and diagnostic internal phototherapy and illumination of intracorporeal tissues and organs. More specifically, the invention concerns an artificial intelligence-enhanced medical device configured to deliver precision light to internal tissues via routine clinical access routes, including without limitation nasogastric and orogastric tubes, rectal probes, endotracheal tubes, and umbilical or other vascular catheters. The system includes the device operatively coupled to a controller and data processing unit adapted for therapeutic and diagnostic purposes with real-time sensor-informed adaptive control.

The invention further relates to methods for diagnosing and treating hyperbilirubinemia (Maisels and McDonagh, 2008; Kale et al., 2013), including diagnosis of neonatal jaundice, using the intracorporeal phototherapy device (Mills and Tudehope, 2001; Kumar, Chawla and Deorari, 2011; IEC, 2020).

### BACKGROUND OF THE INVENTION

Phototherapy for neonatal jaundice is usually delivered externally (Stokowski, 2011), which limits efficacy in extremely low birth weight neonates weighing about 500 to 1500 grams (Arnold, Pedroza and Tyson, 2014), where physiological and anatomical constraints reduce exposure area and increase the risk of therapy interruption (Maisels and McDonagh, 2008; Kemper et al., 2022; Pettersson et al., 2022). External phototherapy may compromise the protective barrier of the skin as reported in the clinical literature (Özkan et al., 2003; Brewster et al., 2010; Wang et al., 2021) and requires safeguards against phototoxicity (El-Abdin et al., 2012; Oláh et al., 2013; Gómez-Meda et al., 2014; Mesbah-Namin et al., 2017; Schoor et al., 2020; Bulut, Erek and Duruyen, 2022) and overheating in accordance with recognized infant phototherapy equipment standards (IEC, 2020; Kemper et al., 2022). External systems may impair parent-infant bonding, whereas home-appropriate solutions have been shown to support parent-infant bonding and reduce stress (Pettersson et al., 2022). Clinical challenges include suboptimal dose control, limited responsiveness to bilirubin trends, and disruption of developmental care routines, while avoiding over and undertreatments and achieving rapid reduction of excess bilirubin is time-critical to prevent unnecessary hospital stay, bilirubin encephalopathy or death (Özkan et al., 2003; Rennie, Beer and Upton, 2019; Kemper et al., 2022).

Existing technologies, including CN 107982641 A, WO 2021/252639 A1, and US 2004/073278 A1, disclose internal illumination but do not disclose integration of therapeutic emitters and diagnostic sensors within routinely placed, dedicated-purpose clinical tubes such as endotracheal tubes, nasogastric tubes, rectal probes, and umbilical catheters, together with a proximal dual-branch interface in which one branch provides the control, data, power and optical connection while the second branch is route-specific: a syringe-or pump-compatible feeding connector for enteral routes, a ventilator breathing-system connector for endotracheal routes, or an infusion hub for vascular routes. Nor do they teach closed-loop artificial-intelligence control of dose based on in situ reflectance and thermal feedback. As a result, they neither provide truly concurrent therapeutic and diagnostic capabilities with real-time data-driven adaptability nor are they structurally or functionally optimized for uninterrupted neonatal workflows, including the constraints of extremely low birth weight care.

US 2004/0073278 A1 discloses a device and method for providing therapeutic illumination to internal organs and tissues, including blood, via an intracorporeally employed light-emitting implant intended for bilirubin photodecomposition. In that disclosure, one or more external light emitters are coupled to the implant by dedicated optical fibers that act as light-transmitting conduits and deliver light to the implant's illumination surface. The reference does not disclose integration of therapeutic emitters and diagnostic sensors within routinely placed clinical tubes, real-time sensor-informed artificial-intelligence control of dose, or a proximal dual-branch interface adapted to enteral, airway, vascular or other routes.

Accordingly, there is a critical and unmet clinical need for an integrated internal multi-route AI-driven light-based therapeutic and diagnostic platform embedded in standard medical care tubes that minimizes invasiveness, enables real-time therapy light modulation, enhances diagnostic inference, and supports uninterrupted care delivery.

### SUMMARY OF THE INVENTION

The device according to the invention is designed to deliver precision-controlled internal phototherapy for neonates, children, and adults (Kemper et al., 2022). The device may provide multiple therapeutic and diagnostic functions, including systemic bilirubin reduction through photoisomerization, trend evaluation with real-time monitoring, and enhanced visualization of therapeutic effects.

The device uses adaptive artificial intelligence algorithms to ensure real-time modulation of phototherapy parameters based on individual physiological feedback from a patient, thereby overcoming limitations of conventional static phototherapy solutions (IEC, 2020; Kemper et al., 2022). Sensor-informed control can incorporate optical reflectance, thermal and leakage-current signals to maintain efficacy while respecting safety envelopes defined for infant phototherapy equipment directives (IEC, 2020).

The device is suitable for coupling to external monitoring devices, including transcutaneous bilirubinometers, and its distinctive aspect is artificial-intelligence-controlled internal phototherapy delivered via dual-lumen or multi-route medical tubing.

A proximal dual-branch external interface separates a control, data, power and optical branch from a sterile or non-sterile route-specific functional branch—such as a pump-compatible feeding connector for enteral routes, a ventilator breathing-system connector for endotracheal routes, or an infusion hub for vascular routes—thereby preserving uninterrupted care while maintaining functional isolation between the branches. Power may be supplied by direct wiring, inductive coupling, or an integrated rechargeable battery, with isolation and power management in the controller to enforce safety limits and uninterrupted operation. The device may function independently of or in combination with external phototherapy devices, thereby enhancing treatment outcomes and expanding the scope of clinical applications (Mills and Tudehope, 2001; Maisels and McDonagh, 2008; Kumar, Chawla and Deorari, 2011; Kemper et al., 2022).

Unlike prior intracorporeal light conduits that require dedicated probes, the device co-opts routine access tubes including nasogastric and orogastric tubes, endotracheal tubes, rectal probes, and umbilical or other vascular catheters, eliminating additional device insertions while enabling closed-loop artificial intelligence dosing, control and in situ diagnostics.

The device may comprise miniaturized fiber optics with an external light source, or embedded light-emitting diodes powered by direct wiring, wireless induction, or an internal rechargeable battery.

Light emission can be localized or distributed along the tube length to provide customizable and optimized coverage, with spectral targeting that includes the bilirubin-efficient blue band (Mills and Tudehope, 2001; Kumar, Chawla and Deorari, 2011; 2022; Lamola et al., 2013; Lamola and Russo, 2014; Sherbiny et al., 2016) and safety governed by recognized infant phototherapy equipment standards (IEC, 2020).

### BRIEF DESCRIPTION OF THE DRAWINGS

The drawings illustrate exemplary embodiments and functional diagrams of the invention. The drawings are schematic and not to scale. Like reference numerals denote like elements across the figures. Features shown in any figure can be combined with features of any other figure unless stated otherwise.

FIG. 1 is a functional block diagram showing the principal components of the device, including a flexible medical tube, light sources, sensors, power supply unit, control unit, and communication interface, optionally integrated in a proximal head unit.

FIG. 2 illustrates a representative enteral (nasogastric/orogastric) embodiment incorporating embedded emitters and sensors with a bifurcated proximal interface that separates a functional feeding branch from control, data, power and optical connection branch.

FIG. 3 illustrates a rectal embodiment configured for transmucosal illumination and feedback sensing through integrated light sources and sensors.

FIG. 4 illustrates an endotracheal embodiment configured for intraluminal illumination of airways and pulmonary tissues while maintaining ventilatory functionality.

FIG. 5 illustrates an umbilical venous catheter embodiment configured for intraluminal illumination of vessels.

FIG. 6 is a functional diagram of the artificial-intelligence feedback and adaptive-control loop, including representative sensor input, AI engine, decision logic, optical control, and safety feedback stages.

FIG. 7 is a schematic overview of an exemplary therapeutic and diagnostic system integrating the intracorporeal illumination device with external processing, monitoring, and safety management units within a clinical environment such as a neonatal intensive care unit.

### DETAILED DESCRIPTION

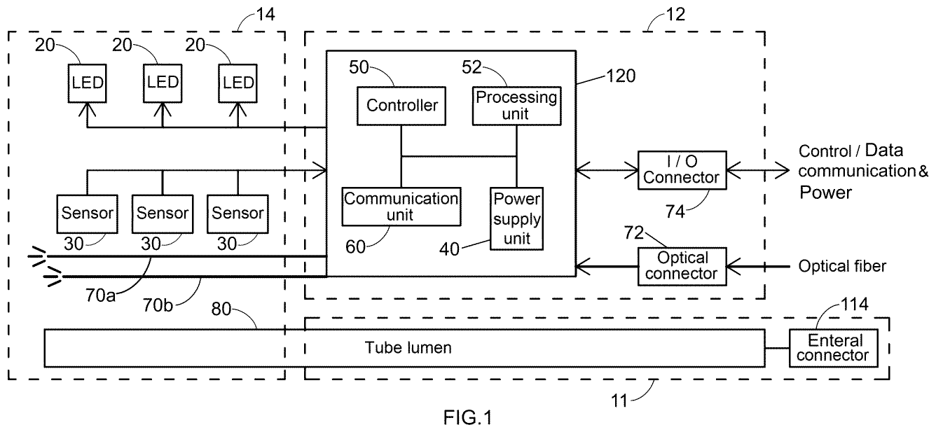

As depicted in FIG. 1, the device according to the present invention comprises a flexible medical tube 10, a plurality of light sources 20, one or more sensors 30, a control unit 50, a preprocessing unit 52 and a communication unit 60. In a preferred embodiment, the device further comprises a head unit 120 arranged at proximal end of the flexible medical tube 10, said head unit 120 being configured to accommodate the control unit 50, the preprocessing unit 52, the communication interface 60, and optionally, an integrated power supply unit 40. The integrated power supply unit 40 may comprise a battery, particularly a rechargeable battery.

In one embodiment, the device may be powered by an external power supply through electric wires.

The medical tube 10 is formed from biocompatible, medical-grade elastomers or polymers, including silicone and polyurethane, selected to provide flexibility, kink resistance, and long-term tissue compatibility (“ISO 10993-1:2018 Biological evaluation of medical devices —Part 1: Evaluation and testing within a risk management process,” 2018). The tube 10 may include radiopaque markers and graduated length markings to assist placement and verification. The tube 10 may be adapted for multiple clinical access routes into a patient's body, including the airways, nasogastric and orogastric routes, rectal routes, and vascular routes, for example umbilical venous catheters referred to as UVC and umbilical arterial catheters referred to as UAC, with other possible access routes as clinically indicated.

The medical tube 10 has two branches at a proximal end thereof. A first branch 11 is a part of the clinical tube serving as an access route for administering clinical materials (e.g. air, drug, medical solution, etc.) to a patient and/or drain materials (e.g. blood, body fluid, etc.) from a patient through a respective connector 114 for the respective application during a clinical treatment. A second branch 12 is used for connecting the device to external devices for providing phototherapy and diagnostic functions during the clinical treatment. The second branch 12 may also be used for data and energy transmission between the device and one or more external devices, including control means, data processing means, power supplying means and/or light producing means.

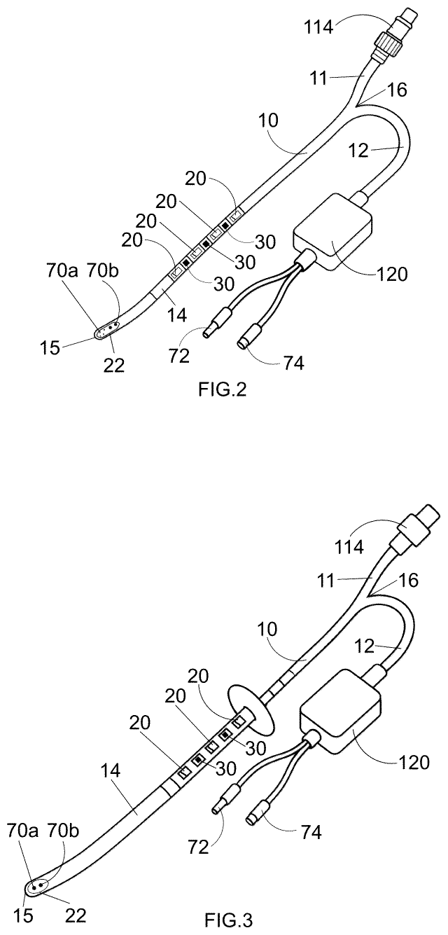

A common section 14 of the medical tube 10 extending from the distal end 15 of the tube 10 to the position 16 of the tube 10 where the tube 10 itself branches (see FIGS. 2 to 5). In this common section 14, the medical tube 10 incorporates phototherapy and diagnostic components, in particular the control, data and power lines, an optional optical fiber 70, the light emitting diodes 20 and the sensors 30, within the tube wall so that these components be physically isolated and insulated from one or more lumens 80 of the medical tube 10.

For connecting the device to said external means, in one embodiment of the device, the head unit 120 is equipped with one or more of an optical connector 72 for coupling to an optical fiber, a wired data communication interface connector for receiving control data and transmitting sensor data, and a power connector for receiving a power line.

In one embodiment, the wired control and data communication interface connectors with the power connector may be integrated into a single I/O connector 74, such as a USB connector.

In one embodiment, the device may comprise a wireless communication unit 60, wherein the phototherapy control data are received from, and the diagnostic data (i.e. sensor data) are transmitted to the external control and data processing means in a wireless manner, for example, via radio communication.

When the device is adapted for phototherapy by using an external optical fiber 70, the head unit 120 may further comprise an optical coupler and an optical control logic for providing the required optical pattern of the optical fiber illumination during phototherapy, on the basis of control information received from the control unit 50. The optical coupler may comprise an optical splitter for splitting the incoming optical fiber into multiple optical fibers 70a, 70b routed within the tube wall.

In some embodiments, the tube wall may include one or more optically clear windows that accommodate the light sources 20, i.e. the LEDs, and the sensors 30 without impeding the main lumen 80 of the tube 10 used for the clinical treatment. A distal tip of the tube 10 may be atraumatic and may include one or more side apertures 22, sized for feeding or drainage material through the lumen 80 of the clinical tube 10 as applicable to the specific clinical access route.

When an optical fiber 70 is used for phototherapy illumination, either alone or in combination with LEDs embedded in the tube wall, the light emitting tip of the optical fiber(s) 70a, 70b is arranged in the one or more side apertures 22 of the tube 10.

As described above, the device of the invention combines the functions of a conventional medical tube with phototherapy and diagnostic functions for providing an integrated clinical tool to perform various clinical treatments and intracorporeal phototherapy (and diagnostics) simultaneously.

### Nasogastric and Orogastric Route

A dual-function architecture of the device according to the invention preserves uninterrupted enteral feeding or medication delivery through the main lumen of the medical tube, while the additional internal phototherapy, control, data and power connection lines are delivered to the tube through a separate structure (IEC, 2020; Kemper et al., 2022).

In one embodiment, shown in FIG. 2, the light sources 20 are micro-scale emitters embedded within the wall of the tube 10, arranged to produce a controlled illumination field along segments of the oral and nasal cavities, the esophagus, and the stomach, with a fiber-optic light-delivery path optionally terminating in the same distal illumination regions (Kumar, Chawla and Deorari, 2022). In another embodiment, when an external light emitting unit is used, an optical fiber 70 is delivered to the tube 10 through an optical connector 72 and a head unit 120, then it is routed within the tube wall to the aperture 22 of the tube 10. In this embodiment, the tube wall provides a protected channel for light transmission and for routing of sensor wires so that reflectance, spectral, thermal, and leakage-current sensing are integrated along the illumination segment (Mills and Tudehope, 2001; Maisels and McDonagh, 2008). The proximal end includes a bifurcated external interface that separates a combined control, data, power and optical branch 12 from a clinical feeding/drainage branch 11 that accepts an enteral connector 114 compatible with enteral small-bore connectors conforming to ISO 80369-3, published in Europe as EN ISO 80369-3, commonly referred to in clinical use as ENFit (“ISO 80369-3:2016 Small-bore connectors for liquids and gases in healthcare applications —Part 3: Connectors for enteral applications,” 2016), or similar purpose connectors with design and testing expectations (“ISO 20695:2020 Enteral feeding systems —Design and testing,” 2020). The two branches 11, 12 are combined into a single common section 14 of the tube 10 so that feeding can continue even when phototherapy and diagnostics are active. Spectral targeting may include the bilirubin-efficient blue band and dosing is limited by real-time thermal and reflectance feedback in accordance with clinical guidance and equipment safety requirements (“ISO 10993-1:2018 Biological evaluation of medical devices—Part 1: Evaluation and testing within a risk management process,” 2018; IEC, 2020; Kemper et al., 2022).

### Rectal Probe Route

For the rectal probe embodiment shown in FIG. 3, the tube 10 delivers internal phototherapy via the rectal probe's external surface with the LED light sources 20 positioned to exploit vascular-rich mucosal surfaces for efficient transmucosal absorption. This access route can provide high systemic bilirubin reduction efficiency while keeping external care pathways uninterrupted. When a fiber optic-based light delivery path is used for intracorporeal illumination, optical transmission and sensor feedback routing remain isolated from the main lumen 80 of the tube 10 used as a dedicated pathway for drainage or medication (“IEC 60601-1-8:2006+AMD1:2012+AMD2:2020 CSV Medical electrical equipment—Part 1-8: General requirements, tests and guidance for alarm systems in medical electrical equipment and medical electrical systems,” 2020). The geometry of the distal segment of the medical tube 10 may include circumferential or segmental diffusion holes or apertures 22 through which uniform fiber optic illumination may be produced while a plurality of low-profile sensor zones of the sensors 30 provide reflectance spectroscopy, thermal and leakage-current feedback to the control unit 50 for closed-loop illumination control. Spectral selection and duty cycle are constrained by the same clinical and safety references used for other clinical routes (“ISO 10993-1:2018 Biological evaluation of medical devices—Part 1: Evaluation and testing within a risk management process,” 2018; IEC, 2020).

### Endotracheal Route

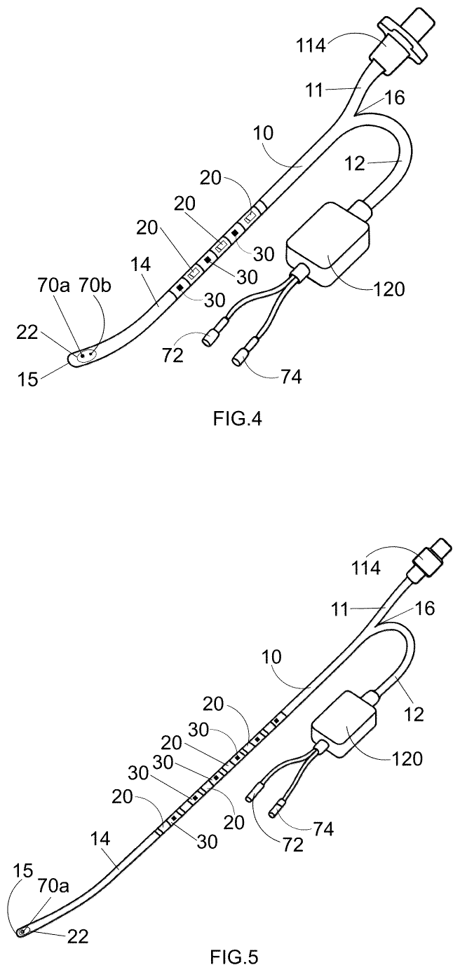

In embodiments for the endotracheal route, shown in FIG. 4, the tube 10 is configured for mucosal, airway and pulmonary parenchyma therapy integration. The light sources 20 and the sensors 30 are embedded in the tube wall to deliver controlled intraluminal light to and across the tracheal mucosa while preserving airway patency and ventilatory performance through the main lumen 80 of the medical tube 10. The illumination segment geometry, angular distribution, and diffusion features are selected so that the effective internal diameter, resistance, and compliance remain within clinical expectations, with connector performance conforming to tracheal tube and breathing system requirements (“ISO 5361:2023 Anaesthetic and respiratory equipment—Tracheal tubes and connectors,” 2023) and related respiratory equipment standards. Sensor feedback provides reflectance spectroscopy together with continuous thermal monitoring and leakage-current detection, enabling the control unit 50 to adjust spectral irradiance, duty cycle, and exposure duration within predetermined infant phototherapy equipment safety limits governing tissue temperature and electrical leakage thresholds (IEC, 2020). Spectral targeting includes the blue efficacy band identified in bilirubin action-spectrum studies (Lamola and Russo, 2014) and dosing aligns with contemporary neonatal guidance (IEC, 2020).

### Umbilical Venous Catheter and Umbilical Arterial Catheter Route

When the access route is intravascular, an umbilical venous catheter, as depicted in FIG. 5, or an umbilical arterial catheter may be used as a basic component of the device. The tube 10 and its integrated light delivery components enable direct illumination of blood within a vessel by means of one or more optical fibers 70a, 70b embedded within the wall of the tube 10 or a plurality of integrated light sources 20 so that circulating bilirubin is exposed efficiently to therapeutic light while maintaining line patency and infusion functionality through the main lumen of the tube 10 (Maisels and McDonagh, 2008; “ISO 10555-1:2023 Intravascular catheters—Sterile and single-use catheters 13 Part 1: General requirements,” 2023). The mechanical characteristics, connection geometry, and material performance of the tube 10 remain consistent with intravascular catheter requirements described in ISO 10555-1. Placement of the sensors 30 allows in-line optical, thermal and leakage-current feedback so that the control unit 50 can determine dose limits and thermal cut-offs defined in the safety envelope in IEC 60601-2-50. This route supports systemic bilirubin reduction mechanisms discussed in the clinical phototherapy literature (Kemper et al., 2022).

### Illumination

The device may be equipped with multiple illumination configurations to enable precise therapy and diagnosis. In a preferred embodiment, a plurality of medical-grade light-emitting diodes arranged in one or more arrays within the wall of the tube 10 are provided as the light sources 20. The light-emitting diodes may include micro-scale or nano-scale emitter technologies recognized in clinical phototherapy practice (Sherbiny et al., 2016; Kumar, Chawla and Deorari, 2022).

In another preferred embodiment, one or more optical fibers 70a, 70b embedded within the wall of the tube 10 may also act as light sources. In this embodiment, the light is generated by an external light emitting unit, the light is transmitted via an optical fiber 70 to the device through an optical connector 72 and coupled into the wall-embedded optical fibers 70a, 70b through an optical coupler formed as a part of the head unit 120 of the second branch 12 of the device, wherein said one or more embedded optical fibers 70a, 70b deliver the light to specific illumination regions along the tube 10 (Mills and Tudehope, 2001). The fiber-optic configuration may include an optical coupling node in the head unit 120, a waveguide-insulated delivery track along the wall of the tube 10, and a distal diffusion segment that produces a controlled illumination field.

The emitted wavelength spectrum is adjustable from about 400 nanometers to about 900 nanometers, spanning from the visible range to the near-infrared range. For hyperbilirubinemia, spectral targeting includes the blue efficacy band identified in foundational bilirubin phototherapy works, and dosing aligns with contemporary neonatal guidance and equipment safety requirements (Maisels and McDonagh, 2008; IEC, 2020; Kemper et al., 2022).

Low-intensity, precisely regulated emission is used to prevent device overheating and tissue injury, and to enable fine-tuned optimal delivery that maximizes efficiency while minimizing overtreatment. Representative internal spectral irradiance levels for intracorporeal illumination are in the order of tens of microwatts per square centimeter, for example about 50 to 100 microwatts per square centimeter, with automatic limits enforced by the controller in accordance with recognized infant phototherapy equipment requirements and site protocols (IEC, 2020; Kemper et al., 2022).

The light-delivery mode is selectable on demand by using an external control unit and communicated to the device through the control unit 50. Fiber-optic delivery supplied by an external emitting unit may be used for high-intensity continuous illumination, while embedded light emitting diodes may be used for localized, energy-efficient light emission (Mills and Tudehope, 2001; Kumar, Chawla and Deorari, 2022).

In a preferred embodiment, artificial-intelligence-controlled adaptive modulation of the illumination is carried out using the onboard control unit 50 that generates the illumination patterns and optimizes energy delivery based on control instructions received from an external control unit and the sensor feedback. The control unit 50, part of head unit 120, also calibrates spectral irradiance at the emission zone, records delivered dose and operating states to an event log, and enforces thermal cut-offs with other limits that define the safety envelope. Depending on the clinical application, the device may produce a uniform field for coverage of a defined anatomical segment or a targeted field for a specific intracorporeal location. The control unit 50 may dynamically and automatically adjust intensity, wavelength, pulse pattern, duty cycle and duration of the illumination based on patient state and safety constraints.

### Control Units

The control unit 50 of the device performs pre-processing of the sensor signals and is further configured to control the operation of the embedded light-emitting diodes. The sensor signals are real-time patient signals measured by the sensors 30 embedded within the wall of the tube 10. The control unit 50 forwards the preprocessed sensor signals to an external control unit that performs artificial-intelligence-driven therapy automation and dynamically adjusts, through the control unit 50 of the device, intensity, wavelength, pulse parameters, and duration of illumination in response to optical reflectance, thermal, and leakage-current feedbacks of the sensors, and maintains efficacy while operating within a standards-based safety envelope.

The onboard control unit 50 may be operated locally via a wired connection or remotely by means of Bluetooth or radio-frequency communication, enabling selection of illumination parameters and operating modes while all safety limits remain enforced on the device.

Diagnostic and operational data may be transmitted by the onboard control unit 50, either via a wired connection or by Bluetooth or radio-frequency communication, to an external clinical monitoring system that may be compliant with HL7, FHIR, or other interoperability frameworks. The onboard control unit 50 comprises a non-transitory computer-readable medium that stores all deterministic and adaptive illumination schemes, including pre-programmed therapy durations, automatic shut-off functions, safety interlocks, dose and event logging, and cybersecurity controls. These safety-critical functions reside exclusively on the device to ensure full authority and uninterrupted operation even in the event of delayed, lost, or unavailable external connectivity.

An external control or supervisory device may store software for visualization, configuration, advisory computations, or data review; however, such external systems do not control illumination, cannot override on-device safety mechanisms, and do not participate in enforcing safety limits. All essential performance and safety functions remain within the onboard control unit 50.

### Power Supply Unit

The device according to the invention is designed for continuous, portable, and autonomous operation with multiple power supply configurations, including direct electrical wiring for uninterrupted hospital-based use, inductive wireless charging for enhanced patient mobility, an integrated rechargeable battery for outpatient or portable therapy and hybrid configuration, integrating multiple ones of the aforementioned power supply configurations and allowing manual or automated switching between them to ensure uninterrupted operation of the device.

The power is provided for the electronic device components by the power supply unit 40, which may be integrated into the head unit 120 of the device in some preferred embodiments. As mentioned before, the device may be powered by a disposable battery, a rechargeable battery or from the mains.

### Diagnostics

The control unit 50 of the device, beside its common electronic, optical and power controlling functions, may also perform certain medical diagnostic functions. It may have various diagnostic preprocessing capabilities, including, but not limited to, determining bilirubin dynamics, generating thermal mapping, measuring spectral reflectance, etc. The results of the preprocessing may be forwarded in a wired or wireless manner to an external processing unit for further data handling, AI-driven evaluation and visualization. The diagnosis results, notifications and alerts may be visualized on a display or presented in audible form via speakers. The device according to the invention, the external control unit, the external data processing unit, the video display, the speakers and the wired or wireless communication tools together form a medical illumination system for diagnostic and therapeutic illumination of intracorporeal organs and tissues, wherein this system provides a further aspect of the present invention.

The diagnostic and therapeutic illumination system according to the present invention may be configured to deliver and perform, among others, one or more of the following medical applications:
Enhanced transillumination for airways, lungs, esophageal, and gastrointestinal visualization
Pneumothorax detection through evaluation of transpleural light transmission
Non-invasive perfusion and oxygenation monitoring via AI-analyzed light absorption differentials
Mucosal tissue assessment and real-time monitoring of oral and airway integrity using AI-assisted reflectance pattern recognition
In situ bilirubin estimation via spectral absorption
Hemoglobin and perfusion assessment using secondary wavelengths
AI-assisted pattern recognition for predictive modeling of disease etiology (e.g., hemolysis, sepsis, liver immaturity) and providing aid for diagnostic categorization

### Example Embodiment

An example embodiment of the device according to the invention delivers blue-green illumination within a range from about 450 nm to about 580 nm. The instantaneous band-integrated irradiance at the emission window is approximately 80 microwatts per square centimeter applied with a duty cycle of 30%, yielding a time-averaged band-integrated irradiance of about 24 microwatts per square centimeter. Real-time thermal feedback enforces a default temperature ceiling at the mucosa-device interface of not more than 37° C. In some embodiments, a user-settable upper limit within a range that may extend up to about 40° C. can be configured, provided that local exposure time and safety margins have been validated for the intended population. In addition, the device may include leakage-current monitoring configured to detect leakage current above a predefined threshold and to initiate a safety shutdown to prevent electrical injury. Modulation parameters are updated every 4 seconds in response to reflectance-derived feedback. Efficacy is governed by the bilirubin response trajectory and not by numeric parity with extracorporeal spectral irradiance, because internal delivery eliminates skin attenuation and shortens optical path length to the target.

### AI Feedback Loop and Adaptive Control Logic

The phototherapy and diagnostic illumination system according to the invention provides an artificial intelligence driven feedback loop and adaptive control logic that delivers precision intracorporeal illumination through dynamic, individualized optimization of the illumination parameters. The system enables highly personalized therapeutic adjustments with real time responsiveness to a patient's clinical condition and physiological feedback. In addition to fine tuning of light intensity, the system may, for example, derive diagnostic insight by recognizing bilirubin elimination patterns associated with breast milk jaundice, breastfeeding jaundice, Gilbert syndrome, glucose-6-phosphate dehydrogenase deficiency, red blood cell disorders, and other conditions indicated by direct or indirect bilirubin elevations, and other metabolic abnormalities.

### Architecture Overview

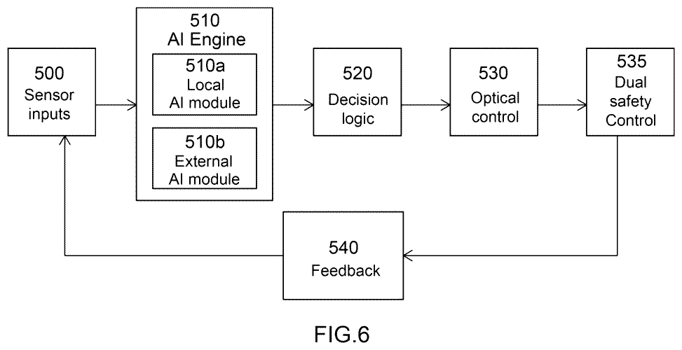

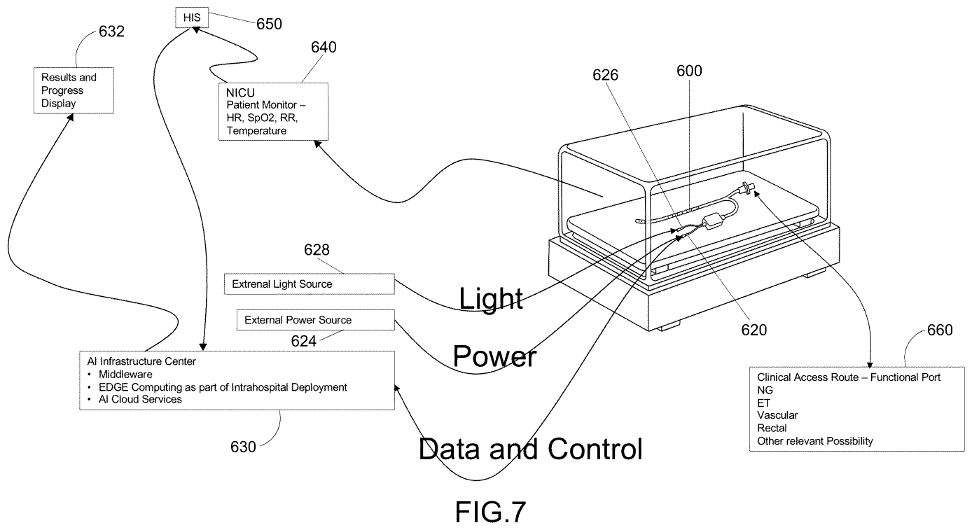

The AI-driven feedback loop and the adaptive control logic form a distributed subsystem within the phototherapy and diagnostic illumination system, and it is in communication with the control unit of the device of the invention. The control unit executes resource-constrained, real-time logic within a small physical footprint, whereas an AI infrastructure center, implemented for example as an on-premise server, an edge gateway or a cloud platform, executes more computationally intensive AI algorithms. As schematically illustrated in FIG. 6, in a sensor input stage 500, the sensor signals received from the one or more sensors are first preprocessed in the preprocessing unit of the head unit and supplied to the control unit of the device. The control unit of the device hosts a local AI and decision module 510a and communicates with an external AI module 510b in the AI Infrastructure Center (see FIG. 7), the modules 510a and 510b together forming an AI engine 510. The local AI and decision module 510a performs real-time decision logic, including rule-based thresholds, safety margins and, in some embodiments, lightweight inference of compact machine-learning models, and provides conservative fallback settings in case the connectivity to the external AI module 510b is lost. The external AI module 510b executes more computationally intensive AI models, including deep-learning architectures trained on larger clinical datasets, in order to estimate bilirubin reduction response, predict treatment outcomes and compute optimized setpoints for illumination intensity, spectral composition and treatment duration. The setpoints provided by the AI engine 510 are applied by a decision layer 520 and an optical control stage 530, both residing in the head unit of the device, while a dual safety controller 535 of the device monitors the sensor signals and enforces predefined safety limits, including a leakage-current and thermal safety envelope at the mucosa-device interface, by limiting or interrupting optical output or electric supply when thresholds are approached or exceeded.

### Sensor Input Stage 500

The device of the invention may perform continuous acquisition of physiological and therapy signals through the sensors embedded in the wall of the medical tube of the device. Representative inputs include light reflectance used to characterize optical absorption, tissue interaction, and bilirubin breakdown efficiency. The sensor functions also include temperature monitoring used to ensure continuous compliance with safe therapeutic temperature ranges. In addition, the device includes leakage-current monitoring configured to detect leakage current above a predefined threshold and to trigger a safety shutdown to prevent direct electrical injury. Based on the sensor data, the illumination system may further estimate unconjugated bilirubin concentration from reflectance spectra or related models to quantify therapy effectiveness and to drive diagnostic trend analysis. Signal quality is maintained by calibration, reference checks, drift detection, artifact rejection, time stamping, and event logging. Time synchronization, serialized data records, and audit-ready logs support traceability and clinical review.

### Artificial Intelligence Engine 510

The artificial intelligence (AI) engine 510 is hosted on an edge computing node in the hospital network or on a governed cloud service, on the one hand, and also on the phototherapy device of the invention, on the other hand. The AI engine 510 receives sensor streams and operational context from the control unit of the device over authenticated encrypted channels, performs inference using validated models, and returns to the device advisory therapy parameters or parameter trajectories matched to the current clinical state. The computing model may include
fuzzy logic that captures subtle physiologic variability and complex elimination patterns,
reinforcement learning agents that refine therapeutic effectiveness while recognizing distinct elimination profiles, and
adaptive neural networks that model patient-specific therapeutic and diagnostic response patterns and identify optical signatures associated with bilirubin metabolism disorders.

Model development, verification, versioning, monitoring, and change control follow the documented medical device software life cycle and the product risk management processes. Because this therapy is not immediate time critical, advisory updates may be near real time with response windows defined by the clinical site in seconds to minutes.

### Decision Logic 520

Decision logic 520 residing in the control unit of the phototherapy device of the invention arbitrates between locally enforced safety rules and advisory parameters from the artificial intelligence engine. Only parameters that satisfy the safety envelope and clinician constraints are applied. The logic performs multi-objective therapy optimization across efficacy, dose, and safety, and it degrades to a validated safe default or to a last known good trajectory if connectivity or confidence is inadequate, and generates clear alerts for clinician attention. Clinician override is always available.

### Optical Control Stage 530

The optical control stage actuates, through the control unit of the device, the embedded LEDs or the fiber-optic emitters to deliver the prescribed therapy. Capabilities may include
real time intensity modulation aligned to diagnostic conditions,
spectral selection within permitted bands, and
pulse-width modulation with bounded duty cycle for precise dosing.

This stage also maintains calibrated spectral irradiance at the emission zone, applies thermal derating in response to sensor inputs, and logs delivered energy and exposure time. The optical control stage is always subordinate to the on-device safety limits.

### Dual Safety Controller 535 and Fault Tolerance

Safety supervision is implemented by a dual safety controller 535 of the phototherapy device of the invention comprising two independent safety channels that operate on the device within the control unit and that are authoritative over any advisory inputs. Each safety channel has its own signal chain for emitter enable, thermal sensing, and leakage-current detection, and its own wiring harness from the proximal control assembly to the illumination segment. The channels supervise each other, and any disagreement or loss of confidence between them causes a latched transition to a safe state that requires deliberate clinician reset. Hardware interlocks are non-bypassable, and a hardware watchdog independently enforces shutdown on timing, logic, temperature, leakage-current, or power anomalies. External systems, including edge and cloud services, cannot raise safety limits or disable interlocks. The design addresses single-fault conditions with two means of patient protection and includes medical-grade isolation of power lines, protective-earth compliance where applicable, current limiting for the emitter path, thermal derating based on local sensor feedback, deterministic shutdown, event logging, and with audible and visual alarm generation.

### Middleware integration layer

Middleware does not perform inference. It brokers messages, normalizes data, maps terminology, enforces consent and privacy rules, and routes observations, orders, alarms, and audit events between the device, the edge node, hospital information systems, and the cloud service. It maintains immutable audit logs and orchestrates store-and-forward when links are impaired. Interoperability with hospital systems is provided through standard clinical data resources for observations, devices, orders, alerts, and audit events.

### Feedback Stage 540 and Learning

The feedback stage 540 residing in the control unit of the phototherapy device evaluates immediate post-actuation sensor responses, validates consistency with expected outcomes, and returns effectiveness metrics to the AI engine 510 for performance monitoring and scheduled retraining under change control. Updates are cryptographically signed, versioned, verified for authenticity and compatibility, and are accepted by the device only when checks pass. Model registry governance and rollback procedures are in place to ensure controlled deployment and rapid reversion when necessary.

### Latency and Resilience

Because dosage adjustments are not immediate time critical, near-real-time inference windows in seconds to minutes are acceptable and are configured by the site. The on-device safety controller guarantees bounded behavior under variable latency or loss of connectivity and ensures uninterrupted therapy within the validated safety envelope. When connectivity is degraded, the device continues with the last applied advisory trajectory or a validated safe default and raises an alert for clinician awareness.

### Security and Privacy

All communication channels are authenticated and encrypted end-to-end. Identifiers are managed under hospital policy with role-based access control and least-privilege principles. Access control, audit logging, cybersecurity controls, and data minimization align with the software life cycle, the product risk management file, and the interoperability framework in use for clinical information systems.

### THERAPEUTIC AND DIAGNOSTIC APPLICATIONS

An exemplary therapeutic and diagnostic system using the intracorporeal illumination device according to the invention is illustrated in FIG. 7, which shows a functional block diagram of the key components of the system. A patient, for example a newborn, may be examined and treated using a phototherapy device 600 according to the invention. The device 600 may comprise a flexible medical tube suitable for nasogastric, umbilical, rectal and other clinically indicated access routes 660. The device 600 may be provided with coupling means 620 for external monitoring and processing, including a secure wired or wireless communication interface that connects the device 600 to a remote AI Infrastructure Center 630 having a dashboard and a display 632. The system exposes a standards-based interoperability interface for the exchange of orders, observations, alerts and audit events, without participating in the on-device safety control path. The coupling means may also be used for supplying power to the phototherapy device 600 from an external power source 624.

The phototherapy device 600 may further be provided with an optical connector 626 for receiving light from an external light source 628 via an optical fiber and to forward the light to the phototherapy device 600.

The phototherapy device 600 may be integrated with a Neonatal Intensive Care Unit 640 for monitoring biological and physical features of a neonatal patient using the sensor data produced by the device 600. The Neonatal Intensive Care Unit 640 may forward the processed biological and physical features of the patient to the Hospital Information System (HIS) 650, which is configured to associate the measurements data with the patient's record and to store the measurements. The HIS 650 may forward the processed clinical context to the AI infrastructure Center 630 to refine illumination pattern selection and energy intensity during the diagnostic or treatment workflows.

The AI infrastructure Center 630 may be configured to operate as defined in the “AI Feedback Loop and Adaptive Control Logic” section above (see FIG. 6). Inference runs off the phototherapy device 600 on an edge or governed cloud engine, the device-resident decision logic applies only advisory parameters that satisfy clinician constraints and the locally enforced safety limits, middleware provides routing and interoperability, and the on-device dual safety channels remain authoritative at all times.

The system shown in FIG. 7 may be optimized for neonatal patients, including preterm infants down to five hundred grams, where conventional external light therapy can be unfeasible or unsafe, and it is equally applicable across the entire newborn population including term infants. While the therapeutic and diagnostic system is optimized for the treatment of neonatal jaundice, its modular light delivery and artificial intelligence-controlled architecture allow adaptation for investigational or adjunct use in other fields such as hepatic support, mucosal diagnostics and photodynamic therapy. Adaptability is enabled by variable catheter sizes, wavelength tuning and route-specific parameter sets, together with a proximal dual-branch external interface that separates the control, data, power and optical branch from a route-specific sterile functional branch so that enteral feeding, ventilatory support or vascular infusion proceed without interruption.

In a further aspect, the present invention relates to a method for diagnosing neonatal jaundice using the phototherapy device according to the present invention. The diagnostic method comprises the following steps:
a) the flexible medical tube of the phototherapy device is inserted into a clinical access route of a neonatal patient;
b) light emission is applied using machine-learning-based modulation by the control unit of the phototherapy device;
c) real-time spectral and thermal data are obtained from the sensors of the phototherapy device;
d) a bilirubin-reduction response curve is estimated by the control unit of the phototherapy device;
e) jaundice level and etiology are classified using a predetermined algorithm by the external data processing unit using a predetermined algorithm; and
f) based on the classification by the external data processing unit, it is determined whether the neonatal patient suffers from clinically significant jaundice that requires further treatment.

In one embodiment of the method, when it is determined that the neonatal patient suffers from non-physiological jaundice, the external data processing unit further generates a treatment recommendation including at least one of phototherapy intensity, duration, or need for exchange transfusion.

Originally developed for neonates, particularly preterm infants requiring bilirubin reduction, the therapeutic and diagnostic system of the invention has been designed for scalability across the full spectrum of newborns and is adaptable for pediatric and adult applications. By embedding light sources and diagnostic sensors directly into medical-grade biocompatible tubing, the system transforms standard clinical tubes into multifunctional therapeutic and diagnostic platforms. The artificial intelligence driven control system enables near real-time adaptive phototherapy with predictive diagnostic support while preserving the primary clinical functions of the tube and maintaining route-specific workflow continuity.

### Safety and Biocompatibility

The phototherapy device is manufactured from non-toxic, BPA-free, medical-grade materials suitable for human use. Material selection and processing follow a biological evaluation plan consistent with recognized biocompatibility requirements, including cytotoxicity, sensitization, irritation, systemic toxicity, genotoxicity, and hemocompatibility where vascular routes are used (IEC, 2020).

The phototherapy device employs passive thermal management features to maintain tissue-safe temperatures at the illumination segment. Real-time thermal sensing provides continuous local feedback to the controller, which applies automatic thermal derating and enforces hard limits so that surface and contact temperatures remain within the validated safety envelope defined for infant phototherapy equipment (Mills and Tudehope, 2001; IEC, 2020; Kumar, Chawla and Deorari, 2022). The device's onboard dual safety controller supervises emitters enable thermal and leakage-current protection and commands a latched safe state with local audible and visual alarms when thresholds are exceeded or when a fault, disagreement, or loss of confidence is detected between independent safety channels (“IEC 60601-1:2005+AMD1:2012+AMD2:2020 CSV Medical electrical equipment—Part 1: General requirements for basic safety and essential performance (Edition 3.2),” 2020; “IEC 60601-1-8:2006+AMD1:2012+AMD2:2020 CSV Medical electrical equipment—Part 1-8: General requirements, tests and guidance for alarm systems in medical electrical equipment and medical electrical systems,” 2020).

The phototherapy device comprises sterile, single-use components designed to minimize infection risk in neonatal intensive care and other clinical or home settings. Sterility is achieved by a validated ethylene oxide or radiation process with a specified sterility assurance level and maintained by a qualified sterile-barrier system and packaging configuration. An Extractable and Leachable (E&L) program and residuals testing verify that process chemicals and sterilant residues remain within acceptable limits after sterilization (“ISO 11135:2014 Sterilization of health-care products—Ethylene oxide—Requirements for the development, validation and routine control of a sterilization process for medical devices,” 2014; “ISO 10993-1: 2018 Biological evaluation of medical devices—Part 1: Evaluation and testing within a risk management process,” 2018; “ISO 11137-1:2025 Sterilization of health care products—Radiation—Part 1: Requirements for development, validation and routine control of a sterilization process for medical devices,” 2025). Instructions for use include handling, storage, and end-of-life disposal of single-use components.

Electrical safety, isolation, and leakage current limits meet general medical-electrical requirements. Electromagnetic emissions and immunity are controlled for clinical use environments, and when configured for home use, the phototherapy device conforms to the additional requirements for home healthcare equipment (IEC, 2020; “IEC 60601-1:2005+AMD1:2012+AMD2:2020 CSV Medical electrical equipment—Part 1: General requirements for basic safety and essential performance (Edition 3.2),” 2020; “IEC 60601-1-8:2006+AMD1:2012+AMD2:2020 CSV Medical electrical equipment—Part 1-8: General requirements, tests and guidance for alarm systems in medical electrical equipment and medical electrical systems,” 2020). Risk controls are implemented under a documented risk-management process, and safety-related software functions are developed and maintained under a medical-device software life-cycle process (“IEC 62304:2006+AMD1:2015 CSV Medical device software —Software life cycle processes (Edition 1.1),” 2015; “ISO 14971:2019 Medical devices—Application of risk management to medical devices,” 2019).

An automatic safety shutdown is executed when thermal or other safety thresholds are reached. Advisory systems external to the device, including edge or cloud services, cannot raise safety limits or bypass interlocks; the on-device safety controller remains authoritative all times. Clinician override is available for non-safety functions, while safety actions and delivered dose are recorded to an immutable audit log for clinical review and traceability (“IEC 60601-1-8:2006+AMD1:2012+AMD2:2020 CSV Medical electrical equipment—Part 1-8: General requirements, tests and guidance for alarm systems in medical electrical equipment and medical electrical systems,” 2020).

### REFERENCES

Arnold, C., Pedroza, C. and Tyson, J. E. (2014) “Phototherapy in ELBW newborns: Does it work? Is it safe? The evidence from randomized clinical trials,” Seminars in Perinatology, 38(7), pp. 452-464. Available at: https://doi.org/10.1053/j.semperi.2014.08.008.

Brewster, D. H. et al. (2010) “Risk of skin cancer after neonatal phototherapy: retrospective cohort study,” Archives of Disease in Childhood, 95(10), p. 826. Available at: https://doi.org/10.1136/adc.2009.179275.

Bulut, O., Erek, A. and Duruyen, S. (2022) “Effects of hyperbilirubinemia on markers of genotoxicity and total oxidant and antioxidant status in newborns,” Drug and Chemical Toxicology, 45(1), pp. 451-455. Available at: https://doi.org/10.1080/01480545.2019.1710182.

El-Abdin, M. Y. Z. et al. (2012) “Phototherapy and DNA changes in full term neonates with hyperbilirubinemia,” Egyptian Journal of Medical Human Genetics, 13(1), pp. 29-35. Available at: https://doi.org/10.1016/j.ejmhg.2011.11.003.

Gómez-Meda, B. C. et al. (2014) “Effects of blue light phototherapy on DNA integrity in preterm newborns,” Journal of Photochemistry and Photobiology B: Biology, 141, pp. 283-287. Available at: https://doi.org/10.1016/j.jphotobiol.2014.09.012.

IEC (2020) “IEC 60601-2-50:2020 Medical electrical equipment—Part 2-50: Particular requirements for the basic safety and essential performance of infant phototherapy equipment,” (IEC 60601-2-50:2020). Available at:

https://webstore.iec.ch/en/publication/64029.

“IEC 60601-1-8:2006+AMD1:2012+AMD2:2020 CSV Medical electrical equipment—Part 1-8: General requirements, tests and guidance for alarm systems in medical electrical equipment and medical electrical systems” (2020), (IEC 60601-1-8 Ed.2.1).

“IEC 60601-1:2005+AMD1:2012+AMD2:2020 CSV Medical electrical equipment—Part 1: General requirements for basic safety and essential performance (Edition 3.2)” (2020), (IEC 60601-1 Ed.3.2).

“IEC 62304:2006+AMD1:2015 CSV Medical device software—Software life cycle processes (Edition 1.1)” (2015), (IEC 62304 Ed. 1.1).

“ISO 5361:2023 Anaesthetic and respiratory equipment—Tracheal tubes and connectors” (2023), (ISO 5361:2023). Available at: https://www.iso.org/standard/ 77497.html.

“ISO 10555-1:2023 Intravascular catheters—Sterile and single-use catheters—Part 1: General requirements” (2023), (ISO 10555-1:2023). Available at:

https://www.iso.org/standard/76494.html.

“ISO 10993-1: 2018 Biological evaluation of medical devices—Part 1: Evaluation and testing within a risk management process” (2018), (ISO 10993-1:2018). Available at:

https://www.iso.org/standard/68936.html.

“ISO 11135:2014 Sterilization of health-care products—Ethylene oxide—Requirements for the development, validation and routine control of a sterilization process for medical devices” (2014), (ISO 11135:2014). Available at: https://www.iso.org/standard/56137.html.

“ISO 11137-1:2025 Sterilization of health care products—Radiation—Part 1: Requirements for development, validation and routine control of a sterilization process for medical devices” (2025), (ISO 11137-1:2025). Available at: https://www.iso.org/standard/81721.html.

“ISO 14971:2019 Medical devices—Application of risk management to medical devices” (2019), (ISO 14971:2019).

“ISO 20695:2020 Enteral feeding systems—Design and testing” (2020), (ISO 20695:2020). Available at: https://www.iso.org/standard/68853.html.

“ISO 80369-3:2016 Small-bore connectors for liquids and gases in healthcare applications—Part 3: Connectors for enteral applications” (2016), (ISO 80369-3:2016). Available at: https://www.iso.org/standard/50731.html.

Kale, Y. et al. (2013) “Effects of phototherapy using different light sources on oxidant and antioxidant status of neonates with jaundice,” Early Human Development, 89(12), pp. 957-960. Available at: https://doi.org/10.1016/j.earlhumdev.2013.09.013.

Kemper, A. R. et al. (2022) “Clinical Practice Guideline Revision: Management of Hyperbilirubinemia in the Newborn Infant 35 or More Weeks of Gestation,” Pediatrics, 150(3). Available at: https://doi.org/10.1542/peds.2022-058859.

Kumar, P., Chawla, D. and Deorari, A. (2011) “Light-emitting diode phototherapy for unconjugated hyperbilirubinaemia in neonates,” Cochrane Database of Systematic Reviews, (12), p. CD007969. Available at: https://doi.org/10.1002/14651858.cd007969.pub2.

Kumar, P., Chawla, D. and Deorari, A. (2022) “Light-emitting diode phototherapy for unconjugated hyperbilirubinaemia in neonates,” Cochrane Database of Systematic Reviews, (12), p. CD007969. Available at: https://doi.org/10.1002/14651858.cd007969.pub2.

Lamola, A. A. et al. (2013) “The effect of hematocrit on the efficacy of phototherapy for neonatal jaundice,” Pediatric Research, 74(1), pp. 54-60. Available at: https://doi.org/10.1038/pr.2013.67.

Lamola, A. A. and Russo, M. (2014) “Fluorescence Excitation Spectrum of Bilirubin in Blood: A Model for the Action Spectrum for Phototherapy of Neonatal Jaundice,” Photochemistry and Photobiology, 90(2), pp. 294-296. Available at: https://doi.org/10.1111/php.12167.

Maisels, M. J. and McDonagh, A. F. (2008) “Phototherapy for Neonatal Jaundice,” The New England Journal of Medicine, 358(9), pp. 920-928. Available at: https://doi.org/10.1056/nejmct0708376.

Maisels, M. J. and McDonagh, A. F. (2008) “Phototherapy for Neonatal Jaundice,” The New England Journal of Medicine, 358(9), pp. 920-928. Available at:

https://doi.org/10.1056/nejmct0708376.

Mesbah-Namin, S.A. et al. (2017) “An Increased Genotoxic Risk in Lymphocytes from Phototherapy-Treated Hyperbilirubinemic Neonates,” Iranian Biomedical Journal, 21(3), pp. 182-189. Available at: https://doi.org/10.18869/acadpub.ibj.21.3.182.

Mills, J. F. and Tudehope, D. (2001) “Fibreoptic phototherapy for neonatal jaundice,” Cochrane Database of Systematic Reviews, (1), p. CD002060. Available at:

https://doi.org/10.1002/14651858.cd002060.

Oláh, J. et al. (2013) “Long-term hazards of neonatal blue-light phototherapy,” British Journal of Dermatology, 169(2), pp. 243-249. Available at:

https://doi.org/10.1111/bjd.12335.

Özkan, H. et al. (2003) “Dermal bilirubin kinetics during phototherapy in term neonates,” Acta Pædiatrica, 92(5), pp. 577-581. Available at: https://doi.org/10.1111/j.1651-2227.2003.tb02510.x.

Pettersson, M. et al. (2022) “Home phototherapy of term neonates improves parental bonding and stress: Findings from a randomised controlled trial,” Acta Paediatrica, 111(4), pp. 760-766. Available at: https://doi.org/10.1111/apa.16231.

Rennie, J. M., Beer, J. and Upton, M. (2019) “Learning from claims: hyperbilirubinaemia and kernicterus,” Archives of Disease in Childhood—Fetal and Neonatal Edition, 104(2), p. F202. Available at: https://doi.org/10.1136/archdischild-2017-314622.

Schoor, L. W. E. van der et al. (2020) “Blue LED phototherapy in preterm infants: effects on an oxidative marker of DNA damage,” Archives of Disease in Childhood—Fetal and Neonatal Edition, 105(6), pp. 628-633. Available at:

https://doi.org/10.1136/archdischild-2019-317024.

Sherbiny, H. S. et al. (2016) “High-intensity light-emitting diode vs fluorescent tubes for intensive phototherapy in neonates,” Paediatrics and International Child Health, 36(2), pp. 127-133. Available at: https://doi.org/ 10.1179/2046905515y.0000000006.

Stokowski, L. A. (2011) “Fundamentals of Phototherapy for Neonatal Jaundice,” Advances in Neonatal Care, 11(NA;), pp. S10-S21. Available at:

https://doi.org/10.1097/anc.0b013e31822ee62c.

Wang, J. et al. (2021) “Challenges of phototherapy for neonatal hyperbilirubinemia (Review),” Experimental and Therapeutic Medicine, 21(3), p. 231. Available at: https://doi.org/10.3892/etm.2021.9662.

1. A device for clinical treatment and phototherapy of intracorporeal organs or tissues, the device comprising:
a flexible medical tube for clinical treatment, the tube having a wall formed of a medical-grade biocompatible material, the tube further comprising a main lumen configured to provide a clinical access route for at least one of therapeutic, diagnostic, feeding, drainage, ventilation, vascular, or excretory procedures;
a phototherapy and diagnostic means, comprising:
a plurality of light sources embedded in and arranged along a section of the wall of the medical tube, the light sources being configured to produce light within a wavelength range of about 400 nanometers to about 900 nanometers according to a predetermined or adaptive illumination pattern, illumination mode, and light intensity;
one or more sensors embedded in the wall of the medical tube, the one or more sensors including at least one of an optical spectrophotometer, a photodiode, a thermistor, and a leakage-current sensor;
a control unit operatively coupled to the light sources and to the one or more sensors and configured to control the illumination mode, the illumination pattern, and the light intensity of the emitted light at least based on signals received from the one or more sensors;
a communication unit operable to couple the control unit to external data processing and control units; and
a power supply unit configured to power the light sources, the one or more sensors, the control and communication units through electric wires, wherein said wires are routed within the tube wall and insulated from the main lumen of the medical tube; and
wherein at a proximal end of the device, the tube comprises a first branch for clinical access to a patient and a second branch for providing at least control, data and power connectivity for the device and optionally optical connectivity via a fiber-optic connector, the first and second branches being combined into a single tube section extending from a branching position to a distal end of the medical tube.

2. The device of claim 1, wherein the control unit and the communication unit are disposed in or operatively coupled to a head unit arranged along said second branch of the device.

3. The device of claim 1, wherein the light sources comprise one or more arrays of light-emitting diodes, the diodes including micro-scale or nano-scale light emitters.

4. The device of claim 3, wherein the light sources further comprise an optical fiber in optical communication with an external light-emitting unit via a fiber-optic connector arranged on the second branch of the device, said optical fiber being routed within the wall of the tube to an aperture of the tube at the distal end thereof.

5. The device of claim 1, wherein the power supply unit is selected from the group consisting of a rechargeable battery, direct wiring to an external power supply, and an inductive wireless power supply.

6. The device of claim 1, wherein the communication unit comprises a wireless interface.

7. The device of claim 6, wherein the wireless interface conforms to at least one of Bluetooth Low Energy (BLE), Wi-Fi, or other medical radio standards, and the communication unit further comprises a wired communication interface.

8. The device of claim 1, wherein the control unit is configured to:
receive real-time input from the one or more sensors; and
dynamically modulate the light emission based on physiological and spectral feedback received from the one or more sensors.

9. The device of claim 1, further comprising an artificial-intelligence-controlled safety mechanism configured to monitor thermal and leakage-current thresholds and to automatically suspend therapy upon detection of unsafe tissue exposure when a thermal or electrical threshold exceeds a predetermined value.

10. The device of claim 1, wherein the medical tube is selected from a group consisting of a nasogastric tube, an orogastric tube, a rectal tube, an endotracheal tube, an umbilical venous catheter, and an umbilical arterial catheter.

11. The device of claim 6, wherein the communication unit is configured to transmit diagnostic and operational data to an external clinical control and monitoring system.

12. The device of claim 1, wherein the control unit is configured to estimate a total serum bilirubin concentration based on signals from the optical spectrophotometer sensors.

13. The device of claim 1, wherein the control unit comprises a trained neural network implemented using machine-learning models including at least one of supervised regression models, classification algorithms, or deep-learning architectures, the neural network being trained on neonatal intensive care datasets and other relevant patient datasets and configured, using input from the one or more sensors and optionally patient data received via the communication unit, to detect and differentiate pathophysiological bilirubin profiles in a neonatal patient and to classify jaundice severity and etiology.

14. The device of claim 1, wherein the one or more sensors comprise at least one temperature sensor arranged to measure a temperature at a mucosa-device interface, and wherein the control unit is configured to modulate or suspend the light emission so as to maintain the measured temperature at or below about 37 degrees Celsius, and to allow a user-adjustable safety range defined between about 36 and about 40 degrees Celsius.

15. The device of claim 1, wherein the device further comprises leakage-current monitoring circuitry configured to detect leakage current above a predetermined threshold and to cause the control unit to suspend or terminate the light emission upon such detection.

16. A system for therapeutic and diagnostic illumination of intracorporeal parts of a body, the system comprising:
a device for clinical treatment and phototherapy of intracorporeal organs or tissues as recited in claim 1;
an external control and data processing unit;
a wireless communication interface for communicating with the communication unit of the device as recited in claim 1; and
a display.

17. The system of claim 16, wherein the system is installed in a neonatal intensive care unit configured to monitor the biological and physical properties of a neonatal patient and to provide medical data relating to the neonatal patient to the phototherapy device.

18. A method for diagnosing neonatal jaundice using the phototherapy device of claim 1, the method comprising:
a) inserting the flexible tube of the phototherapy device into a clinical access route of a neonatal patient;
b) performing light emission using machine-learning-based modulation by the control unit of the phototherapy device;
c) acquiring real-time spectral and thermal data from the sensors of the phototherapy device;
d) estimating a bilirubin reduction response curve by the control unit of the phototherapy device;
e) classifying jaundice level and etiology, by means of an external data processing unit, using a predetermined algorithm; and
f) determining, by said external data processing unit and based on the classification, whether the neonatal patient suffers from jaundice.

19. The method of claim 18, further comprising a step of generating, by the external data processing unit, a treatment recommendation including at least one of phototherapy intensity, duration, or need for exchange transfusion when it is determined that the neonatal patient suffers from non-physiological jaundice.
